(19) United States
(12) Patent Application Publication
(10) Pub. No.: US 2024/0161930 A1
(43) Pub. Date: May 16, 2024

(54) PREDICTION METHOD USING STATIC AND DYNAMIC DATA

(71) Applicant: VUNO Inc., Seoul (KR)

(72) Inventors: Yunseob SHIN, Hwaseong-si (KR); Yunwon TAE, Anyang-si (KR); Kyungjae CHO, Seongnam-si (KR); Jaewoo CHOI, Seoul (KR)

(21) Appl. No.: 18/231,110

(22) Filed: Aug. 7, 2023

(30) Foreign Application Priority Data
Nov. 16, 2022 (KR) 10-2022-0153549

(51) Int. Cl. G16H 50/30; G16H 50/50; G16H 50/70

(52) U.S. Cl. CPC G16H 50/30; G16H 50/50; G16H 50/70

(57) ABSTRACT

Disclosed is a method for generating a prediction result by using static data and dynamic data according to an exemplary embodiment of the present disclosure. Specifically, according to the present disclosure, a computing device generates an integrated feature vector from static data and dynamic data of input data by using an artificial neural network model. The computing device generates a dynamic feature vector from the dynamic data of the input data by using the artificial neural network model. The computing device generates a final prediction result of the artificial neural network model based on the integrated feature vector and the dynamic feature vector.

### CROSS-REFERENCE TO RELATED APPLICATIONS

This application claims priority to and the benefit of Korean Patent Application No. 10-2022-0153549 filed in the Korean Intellectual Property Office on Nov. 16, 2022, the entire contents of which are incorporated herein by reference.

### BACKGROUND

### Technical Field

The present disclosure relates to a method for generating a prediction result by using static data and dynamic data.

### Description of the Related Art

In recent years, with the development of machine learning, such as deep learning, the study of artificial neural network models that generate prediction results from time series data such as patient's biometric data has been widely conducted.

Among them, attempts have been made to perform predictions using static data that contains relatively unchanged information in time series data together, in addition to a method for performing prediction by using dynamic data that changes according to each point of view in time series data.

Korean Patent Unexamined Publication No. 2021-0028554 discloses a method and an apparatus for training an AI model by using user's time series behavior data.

### BRIEF SUMMARY

However, a method of using the static data in the related art together is just a method for converting the static data by using a simple method such as embedding, and combining the static data with the dynamic data, and inputting the static data and the dynamic data which are combined into the artificial neural network model, so there is a problem in that characteristics of the static data cannot be reflected well. Therefore, there is a demand of the art on a method for performing a prediction result for data by better reflecting the characteristics of the static data.

The various embodiments of the present disclosure have been made in an effort to generate an integrated feature vector and a dynamic feature vector from static data and dynamic data of input data, and generate a final prediction result for the input data based thereon.

The various embodiments of the present disclosure include a method for generating feature vectors from static data and dynamic data, respectively by using an artificial neural network model, and generating a final prediction result based on the generated feature vectors.

Meanwhile, the technical benefits achieved by the present disclosure is not limited to the above-mentioned technical benefits, and various technical benefits can be included within the scope which is apparent to those skilled in the art from contents to be described below.

Further exemplary embodiments are disclosed in the present disclosure.

An exemplary embodiment of the present disclosure provides a method for generating a prediction result by using static data and dynamic data. The method may include: generating an integrated feature vector from static data and dynamic data of input data by using an artificial neural network model; generating a dynamic feature vector from the dynamic data of the input data by using the artificial neural network model; and generating a final prediction result of the artificial neural network model based on the integrated feature vector and the dynamic feature vector.

In an exemplary embodiment, the dynamic data may include time-series biometric data, and the static data may include information related to a patient other than the time-series biometric data.

In an exemplary embodiment, the generating of the integrated feature vector may include identifying a group to which the static data of the input data belongs, generating a static feature vector from the static data of the input data, obtaining an inter-group adjacent matrix based on dynamic data information corresponding to the identified static data group, and generating the integrated feature vector based on a computation of the static feature vector and the adjacent matrix.

In an exemplary embodiment, the identifying of the group to which the static data of the input data belongs may include categorizing information included in the static data, and identifying one or more groups to which the static data belongs based on the categorized information.

In an exemplary embodiment, the generating of the static feature vector from the static data of the input data may include generating a multi-hot encoding vector based on the identified group information.

In an exemplary embodiment, the adjacent matrix may be generated based on computing a dynamic data distribution for the dynamic data corresponding to the identified static data group, computing a distance between the static data groups based on the dynamic data distribution, and comparing the inter-group distance and a predetermined threshold distance.

In an exemplary embodiment, the generating of the dynamic feature vector from the dynamic data of the input data by using the artificial neural network model may include preprocessing the dynamic data of the input data, and generating a dynamic feature vector based on the preprocessed dynamic data.

Another exemplary embodiment of the present disclosure provides a computer-readable storage medium having a computer program stored therein, in which the computer program which allows operations of generating a prediction result by using static data and dynamic data to be performed. The operations may include: an operation of generating an integrated feature vector from static data and dynamic data of input data by using an artificial neural network model; an operation of generating a dynamic feature vector from the dynamic data of the input data by using the artificial neural network model; and an operation of generating a final prediction result of the artificial neural network model based on the integrated feature vector and the dynamic feature vector.

Still another exemplary embodiment of the present disclosure provides a computing device of generating a prediction result by using static data and dynamic data. The computing device may include: at least one processor; and a memory, and the at least one processor may be configured to generate an integrated feature vector from static data and dynamic data of input data by using an artificial neural network model, generate a dynamic feature vector from the dynamic data of the input data by using the artificial neural network model, and generate a final prediction result of the artificial neural network model based on the integrated feature vector and the dynamic feature vector.

According to an exemplary embodiment of the present disclosure, a method for generating a prediction result for input data by using both static characteristics and dynamic characteristics of the input data can be provided. For example, according to an exemplary embodiment of the present disclosure, the prediction result is generated by using both dynamic characteristics of a time-series biometric data part and static characteristics of other data parts in biometric information of a patient to generate a better prediction result for the input data.

### BRIEF DESCRIPTION OF THE SEVERAL VIEWS OF THE DRAWINGS

The following accompanying drawings are only some of the exemplary embodiment of the present disclosure so as to be used for describing the exemplary embodiment of the present disclosure, and in the technical field of the present disclosure, those (hereinafter, referred to as “normal technician”) skilled in the technical field of the present disclosure can obtain other drawings based on the drawings without an effort to reach a new disclosure.

FIG. 1 is a block diagram of a computing device for predicting cardiac arrest according to an exemplary embodiment of the present disclosure.

FIG. 2 is a schematic view illustrating a network function according to an exemplary embodiment of the present disclosure.

FIG. 3 is a flowchart illustrating a process of generating a final prediction result from input data according to an exemplary embodiment of the present disclosure.

FIG. 4 is a flowchart illustrating a process of generating a static feature vector according to an exemplary embodiment of the present disclosure.

FIG. 5 is a conceptual view illustrating static data and dynamic data of time-series data according to an exemplary embodiment of the present disclosure.

FIG. 6 is a conceptual view illustrating an adjacent matrix depending on the distribution of the dynamic data according to an exemplary embodiment of the present disclosure.

FIG. 7 is a simple and normal schematic view of an exemplary computing environment in which the exemplary embodiments of the present disclosure may be implemented.

### DETAILED DESCRIPTION

The present disclosure discloses a method for generating an integrated feature vector and a dynamic feature vector from static data and dynamic data of input data by using an artificial neural network model, and generating a final prediction result based thereon.

Various embodiments will now be described with reference to drawings. In the present specification, various descriptions are presented to provide appreciation of the present disclosure. However, it is apparent that the embodiments can be executed without the specific description.

“Component,” “module,” “system,” and the like which are terms used in the specification refer to a computer-related entity, hardware, firmware, software, and a combination of the software and the hardware, or execution of the software. For example, the component may be a processing process executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

Moreover, the term “or” is intended to mean not exclusive “or” but inclusive “or.” That is, when not separately specified or not clear in terms of a context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in this specification designates and includes all available combinations of one or more items among enumerated related items.

Further, it should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in this specification and the claims.

In addition, the term “at least one of A or B” should be interpreted to mean “a case including only A,” “a case including only B,” and “a case in which A and B are combined.”

Those skilled in the art need to recognize that various illustrative logical blocks, configurations, modules, circuits, means, logic, and algorithm steps described in connection with the embodiments disclosed herein may be additionally implemented as electronic hardware, computer software, or combinations of both sides. To clearly illustrate the interchangeability of hardware and software, various illustrative components, blocks, constitutions, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented embodiments is provided so that those skilled in the art of the present disclosure use or implement the present disclosure. Various modifications to the embodiments will be apparent to those skilled in the art. Generic principles defined herein may be applied to other embodiments without departing from the scope of the present disclosure. Therefore, the present disclosure is not limited to the embodiments presented herein. The present disclosure should be analyzed within the widest range which is coherent with the principles and new features presented herein.

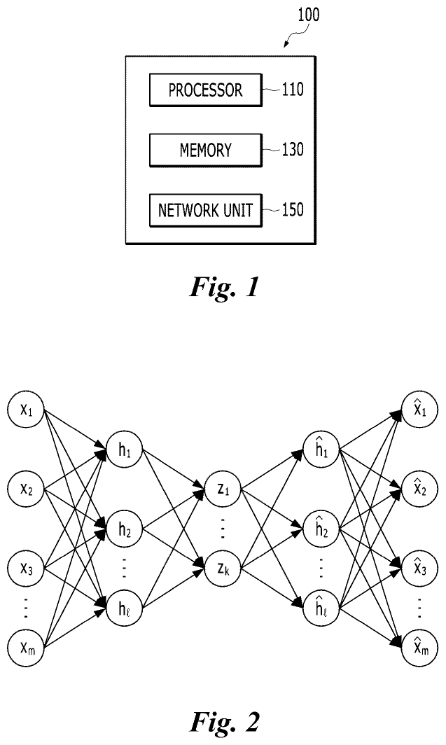

FIG. 1 is a block diagram of a computing device for generating a prediction result by using static data and dynamic data according to an exemplary embodiment of the present disclosure.

A configuration of the computing device 100 illustrated in FIG. 1 is only an example simplified and illustrated. In an exemplary embodiment of the present disclosure, the computing device 100 may include other components for performing a computing environment of the computing device 100, and only some of the disclosed components may constitute the computing device 100.

The computing device 100 may include a processor 110, a memory 130, and a network unit 150.

The processor 110 may be constituted by one or more cores, and include processors for data analysis and deep learning, such as a central processing unit (CPU), a general purpose graphics processing unit (GPGPU), a tensor processing unit (TPU), etc., of the computing device. The processor 110 may read a computer program stored in the memory 130 and process data for machine learning according to an exemplary embodiment of the present disclosure. According to an exemplary embodiment of the present disclosure, the processor 110 may perform an operation for learning the neural network. The processor 110 may perform calculations for learning the neural network, which include processing of input data for learning in deep learning (DL), extracting a feature in the input data, calculating an error, updating a weight of the neural network using backpropagation, and the like.

At least one of the CPU, the GPGPU, and the TPU of the processor 110 may process learning of the network function. For example, the CPU and the GPGPU may process the learning of the network function and data classification using the network function jointly. In addition, in an exemplary embodiment of the present disclosure, the learning of the network function and the data classification using the network function may be processed by using processors of a plurality of computing devices together. In addition, the computer program performed by the computing device according to an exemplary embodiment of the present disclosure may be a CPU, GPGPU, or TPU executable program.

According to an exemplary embodiment of the present disclosure, the processor 110 may obtain input data. The input data may be time-series data, and the time-series data may include dynamic data and static data. In this case, the dynamic data may be data of which value changes for each point of view, e.g., time-series biometric data. Specifically, the dynamic data may include blood pressure, heart rate, etc., of a patient.

The static data may be data which is not changed according to a point of view including information related to the patient other than the dynamic data among the time-series biometric data. Specifically, the static data may include gender, age, and the type of an intensive care unit of the patient.

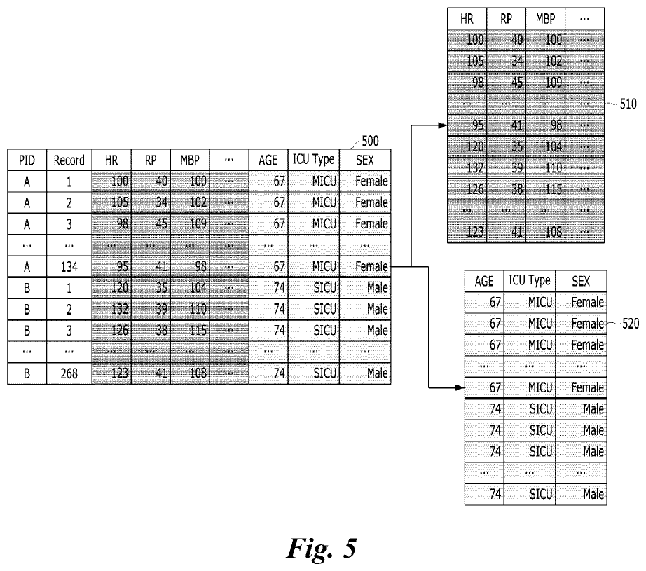

When further described by referring to FIG. 5, input data 500 as the time-series data includes information on respective items of patients A and B. In respect to A and B, data such as heart rate (HR), respiratory rate (RR), average blood pressure (mBP), etc., may correspond dynamic data 510 of which value is changed for each point of view. On the contrary, since information on the age, the gender, and the intensive care unit where each of A and B is positioned in respect to A and B is not data which of which value is changed according to each point of view, the information may correspond to static data 520.

The input data may be data stored in the memory 130 of the computing device 100, may be information measured from a biometric information measurement device connected to the computing device in real time, and may be information received from the network unit 150. However, the present disclosure is not limited to such an acquisition path.

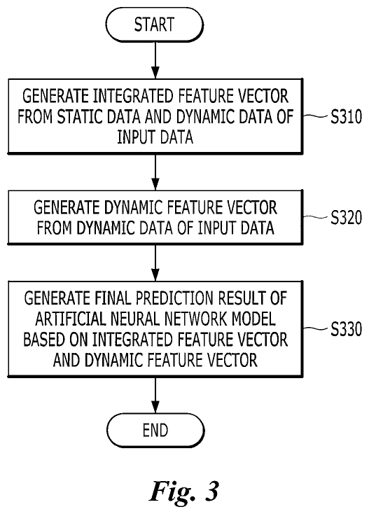

The processor 110 may generate an integrated feature vector from the static data and the dynamic data of the input data. A specific process of generating the integrate feature vector will be described below with reference to FIG. 3.

The processor 110 may generate a dynamic feature vector from the dynamic data of the input data. A specific process of generating the dynamic feature vector will be described below with reference to FIG. 3.

The processor 110 may generate a final prediction result based on the integrated feature vector and the dynamic feature vector by using an artificial neural network model. In the present disclosure, the final prediction result of the artificial neural network model may be whether cardiac arrest may occur in the patient within a predetermined time, and may be an intensive care unit re-entering risk of the patient when the input data is the biometric data of the patient. However, the present disclosure is not limited to an output of the type taken as an example above, and the artificial neural network may be designed to be trained with different types of training data while maintaining a structure in the present disclosure to perform another task.

According to an exemplary embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the present disclosure is not limited thereto.

The network unit 150 according to several exemplary embodiments of the present disclosure may use various wired communication systems, such as a Public Switched Telephone Network (PSTN), an x Digital Subscriber Line (xDSL), a Rate Adaptive DSL (RADSL), a Multi Rate DSL (MDSL), a Very High Speed DSL (VDSL), a Universal Asymmetric DSL (UADSL), a High Bit Rate DSL (HDSL), and a local area network (LAN).

The network unit 150 presented in the present specification may use various wireless communication systems, such as Code Division Multi Access (CDMA), Time Division Multi Access (TDMA), Frequency Division Multi Access (FDMA), Orthogonal Frequency Division Multi Access (OFDMA), Single Carrier-FDMA (SC-FDMA), and other systems.

The network unit 150 according to the exemplary embodiment of the present disclosure may use a predetermined form of a publicly known wired/wireless communication system.

The technologies described in the present specification may be used in other networks, as well as the foregoing networks.

FIG. 2 is a schematic diagram illustrating a network function according to the embodiment of the present disclosure.

Throughout the present specification, the meanings of a calculation model, a nerve network, the network function, and the neural network may be interchangeably used. The neural network may be formed of a set of interconnected calculation units which are generally referred to as “nodes.” The “nodes” may also be called “neurons.” The neural network consists of one or more nodes. The nodes (or neurons) configuring the neural network may be interconnected by one or more links.

In the neural network, one or more nodes connected through the links may relatively form a relationship of an input node and an output node. The concept of the input node is relative to the concept of the output node, and a predetermined node having an output node relationship with respect to one node may have an input node relationship in a relationship with another node, and a reverse relationship is also available. As described above, the relationship between the input node and the output node may be generated based on the link. One or more output nodes may be connected to one input node through a link, and a reverse case may also be valid.

In the relationship between an input node and an output node connected through one link, a value of the output node data may be determined based on data input to the input node. Herein, a link connecting the input node and the output node may have a weight. The weight is variable, and in order for the neural network to perform a desired function, the weight may be varied by a user or an algorithm. For example, when one or more input nodes are connected to one output node by links, respectively, a value of the output node may be determined based on values input to the input nodes connected to the output node and weights set in the link corresponding to each of the input nodes.

As described above, in the neural network, one or more nodes are connected with each other through one or more links to form a relationship of an input node and an output node in the neural network. A characteristic of the neural network may be determined according to the number of nodes and links in the neural network, a correlation between the nodes and the links, and a value of the weight assigned to each of the links. For example, when there are two neural networks in which the numbers of nodes and links are the same and the weight values between the links are different, the two neural networks may be recognized to be different from each other.

The neural network may consist of a set of one or more nodes. A subset of the nodes configuring the neural network may form a layer. Some of the nodes configuring the neural network may form one layer on the basis of distances from an initial input node. For example, a set of nodes having a distance of n from an initial input node may form n layers. The distance from the initial input node may be defined by the minimum number of links, which need to be passed to reach a corresponding node from the initial input node. However, the definition of the layer is arbitrary for the description, and a degree of the layer in the neural network may be defined by a different method from the foregoing method. For example, the layers of the nodes may be defined by a distance from a final output node.

The initial input node may mean one or more nodes to which data is directly input without passing through a link in a relationship with other nodes among the nodes in the neural network. Otherwise, the initial input node may mean nodes which do not have other input nodes connected through the links in a relationship between the nodes based on the link in the neural network. Similarly, the final output node may mean one or more nodes that do not have an output node in a relationship with other nodes among the nodes in the neural network. Further, the hidden node may mean nodes configuring the neural network, not the initial input node and the final output node.

In the neural network according to the embodiment of the present disclosure, the number of nodes of the input layer may be the same as the number of nodes of the output layer, and the neural network may be in the form that the number of nodes decreases and then increases again from the input layer to the hidden layer. Further, in the neural network according to another embodiment of the present disclosure, the number of nodes of the input layer may be smaller than the number of nodes of the output layer, and the neural network may be in the form that the number of nodes decreases from the input layer to the hidden layer. Further, in the neural network according to another embodiment of the present disclosure, the number of nodes of the input layer may be larger than the number of nodes of the output layer, and the neural network may be in the form that the number of nodes increases from the input layer to the hidden layer. The neural network according to another embodiment of the present disclosure may be the neural network in the form in which the foregoing neural networks are combined.

A deep neural network (DNN) may mean the neural network including a plurality of hidden layers, in addition to an input layer and an output layer. When the DNN is used, it is possible to recognize a latent structure of data. That is, it is possible to recognize latent structures of photos, texts, videos, voice, and music (for example, what objects are in the photos, what the content and emotions of the texts are, and what the content and emotions of the voice are). The DNN may include a convolutional neural network (CNN), a recurrent neural network (RNN), an auto encoder, Generative Adversarial Networks (GAN), a Long Short-Term Memory (LSTM), a transformer, a restricted Boltzmann machine (RBM), a deep belief network (DBN), a Q network, a U network, a Siamese network, a Generative Adversarial Network (GAN), and the like. The foregoing description of the deep neural network is merely illustrative, and the present disclosure is not limited thereto.

In the embodiment of the present disclosure, the network function may include an auto encoder. The auto encoder may be one type of artificial neural network for outputting output data similar to input data. The auto encoder may include at least one hidden layer, and the odd-numbered hidden layers may be disposed between the input/output layers. The number of nodes of each layer may decrease from the number of nodes of the input layer to an intermediate layer called a bottleneck layer (encoding), and then be expanded symmetrically with the decrease from the bottleneck layer to the output layer (symmetric with the input layer). The auto encoder may perform a nonlinear dimension reduction. The number of input layers and the number of output layers may correspond to the dimensions after preprocessing of the input data. In the auto encoder structure, the number of nodes of the hidden layer included in the encoder decreases as a distance from the input layer increases. When the number of nodes of the bottleneck layer (the layer having the smallest number of nodes located between the encoder and the decoder) is too small, the sufficient amount of information may not be transmitted, so that the number of nodes of the bottleneck layer may be maintained in a specific number or more (for example, a half or more of the number of nodes of the input layer and the like).

The neural network may be trained by at least one scheme of supervised learning, unsupervised learning, semi-supervised learning, and reinforcement learning. The training of the neural network may be a process of applying knowledge for the neural network to perform a specific operation to the neural network.

The neural network may be trained in a direction of minimizing an error of an output. In the training of the neural network, training data is repeatedly input to the neural network and an error of an output of the neural network for the training data and a target is calculated, and the error of the neural network is back-propagated in a direction from an output layer to an input layer of the neural network in order to decrease the error, and a weight of each node of the neural network is updated. In the case of the supervised learning, training data labelled with a correct answer (that is, labelled training data) is used, in each training data, and in the case of the unsupervised learning, a correct answer may not be labelled to each training data. That is, for example, the training data in the supervised learning for data classification may be data, in which category is labelled to each of the training data. The labelled training data is input to the neural network and the output (category) of the neural network is compared with the label of the training data to calculate an error. For another example, in the case of the unsupervised learning related to the data classification, training data that is the input is compared with an output of the neural network, so that an error may be calculated. The calculated error is back-propagated in a reverse direction (that is, the direction from the output layer to the input layer) in the neural network, and a connection weight of each of the nodes of the layers of the neural network may be updated according to the backpropagation. A change amount of the updated connection weight of each node may be determined according to a learning rate. The calculation of the neural network for the input data and the backpropagation of the error may configure a learning epoch. The learning rate is differently applicable according to the number of times of repetition of the learning epoch of the neural network. For example, at the initial stage of the learning of the neural network, a high learning rate is used to make the neural network rapidly secure performance of a predetermined level and improve efficiency, and at the latter stage of the learning, a low learning rate is used to improve accuracy.

In the training of the neural network, the training data may be generally a subset of actual data (that is, data to be processed by using the trained neural network), and thus an error for the training data is decreased, but there may exist a learning epoch, in which an error for the actual data is increased. Overfitting is a phenomenon, in which the neural network excessively learns training data, so that an error for actual data is increased. For example, a phenomenon, in which the neural network learning a cat while seeing a yellow cat cannot recognize cats, other than a yellow cat, as cats, is a sort of overfitting. Overfitting may act as a reason of increasing an error of a machine learning algorithm. In order to prevent overfitting, various optimizing methods may be used. In order to prevent overfitting, a method of increasing training data, a regularization method, a dropout method of inactivating a part of nodes of the network during the training process, a method using a bath normalization layer, and the like may be applied.

Hereinbelow, a conventional method for performing a prediction by using static characteristics of input data will be described.

A conventional method for performing the prediction by using the static characteristics of data is conducted by a scheme of separating the input data into static data and dynamic data, and then inputting the dynamic data into a dynamic data encoder to generate dynamic feature data, inputting the static data into a static data encoder to generate static feature data, and inputting the dynamic feature data and the static feature data into an integrated data encoder and a classifier to generate a prediction result.

However, in the case of the conventional method, since only a method such as simple embedding is used in reflecting static characteristics of the input data, a relationship between respective information of the static data may not be reflected, so a phenomenon occurs in which accuracy of a generated prediction result deteriorates.

FIG. 3 is a flowchart illustrating a process of generating a final prediction result from input data according to an exemplary embodiment of the present disclosure.

According to FIG. 3, the process of generating the final prediction result from the input data in the present disclosure may be constituted by a step S310 of generating an integrated feature vector from static data and dynamic data of input data, a step S320 of generating the dynamic feature vector from the dynamic data of the input data, and a step S330 of generating a final prediction result of an artificial neural network model based on the integrated feature vector and the dynamic feature vector.

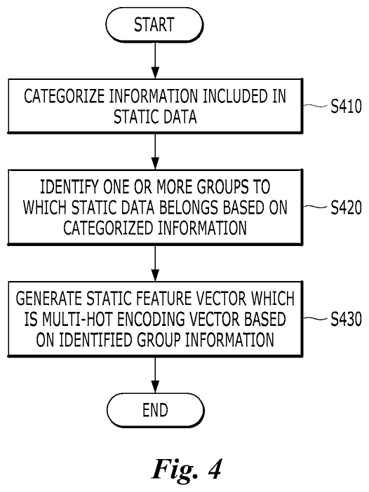

In step S310, the processor 110 may generate the integrated feature vector from the static data and the dynamic data of the input data. The processor 110 may generate a static feature vector from the static data of the input data in order to generate the integrated feature vector. Further, the processor 110 may generate inter-group adjacent matrices based on identifying a group to which the static data of the input data belongs. Thereafter, the processor 110 inputs a vector generated based on a multiplication operation result of the static feature vector and the adjacent matrix into a static data encoder included in the artificial neural network model to generate the integrated feature vector. A specific method for generating each of the static feature vector and the adjacent matrix will be described below with reference to FIG. 4.

In step S320, the processor 110 may generate the dynamic feature vector from the dynamic data of the input data. The processor 110 may preprocess the dynamic data of the input data in order to generate the dynamic feature vector. For example, the processor 110 may regularize a value depending on a time of time-series biometric data such as heart rate (HR), respiratory rate (RR), and blood pressure (mBP) among the biometric data of a patient when the biometric data of the patient is input.

Thereafter, the processor 110 inputs the preprocessed dynamic data into a dynamic data encoder of the artificial neural network model to generate the dynamic feature vector.

In steps S310 and S320, the static data encoder and the dynamic data encoder may have the same structure as a long-short term memory or a transformer for analyzing the time-series data. However, the present disclosure is not limited to the structure of the encoder taken as an example.

In step S330, the processor 110 may generate the final prediction result of the artificial neural network model based on the integrated feature vector and the dynamic feature vector. For example, the processor 110 may generate one vector by concatenating the integrated feature vector which is an output of the static data encoder and the dynamic feature vector which is an output of the dynamic data encoder. Thereafter, the processor 110 inputs one generated vector into a classifier included in the artificial neural network to generate the final prediction result. In this case, a final output of the artificial neural network may become various values including a prediction value for whether cardiac arrest occurs in the patient or a prediction value for whether the patient re-enters an intensive care unit.

In the present disclosure, the output of the artificial neural network is generated based on the integrated feature vector and the dynamic feature vector, and the integrated feature vector includes relationship information between groups of static data and a static feature of the input data. Therefore, through the method for generating the prediction value through the artificial neural network of the present disclosure, relational information between a static data groups of the input data is further reflected, so a remarkable effect of performing more accurate prediction is generated.

FIG. 4 is a flowchart illustrating a process of generating a static feature vector according to an exemplary embodiment of the present disclosure.

According to FIG. 4, the process of generating the static feature vector in the present disclosure may include a step S410 of categorizing information included in the static data, a step S420 of identifying one or more groups to which the static data belongs based on the categorized information, and a step S430 of generating the static feature vector which is a multi-hot encoding vector based on the identified group information.

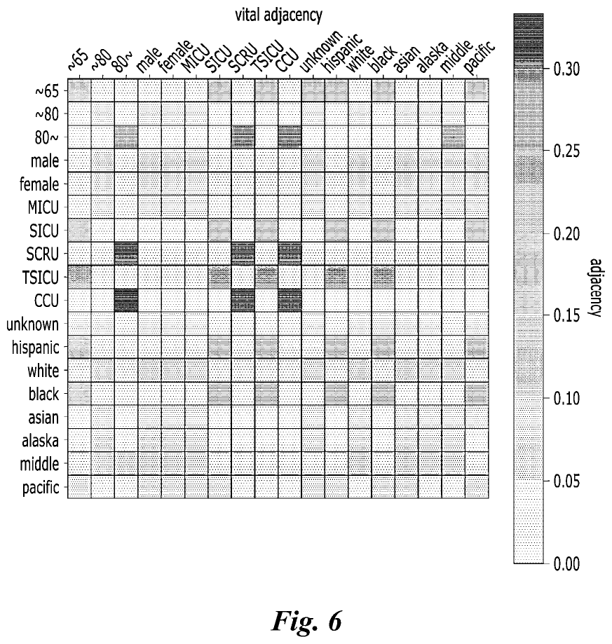

In step S410, the processor 110 may categorize the information included in the static data. For example, when items of the static data are the age of the patient, the gender of the patient, information on the intensive care unit where the patient is positioned, and a race of the patient, the respective items may be categorized. Specifically, referring to FIG. 6, the age item of the patient among the input data may be categorized into [under 65 years old, 65 to 80 years old, and over 80 years old], the information on the intensive care unit may be categorized into [CRSU, CCU, TSICU, and MICU], the gender of the patient may be categorized into [female and male], and the race of the patient may be categorized into [unknown, Hispanic, white, black, Asian, Alaskan, Middle East, and Pacific].

In step S420, the processor 110 may identify one or more groups to which the static data belongs based on the categorized information. Thereafter, the processor 110 may quantify the information of the group and convert the quantified information into a vector. For example, it may be assumed that male patient A who is 87 years old and enters a general intensive care unit (MICU) follows a categorization result for the age of the patient, the gender of the patient, and the information on the intensive care unit where the patient is positioned. In this case, since the patient is 87 years hold, it may be identified that the patient belongs to a group of 80 years old or more, and information on the age of the patient may be quantified to a vector of [0, 0, 1]. Similarly, since it is identified that the intensive care unit where the patient is positioned belongs to the general intensive care unit (MICU), the information on the intensive care unit where the patient is positioned may be quantified to a vector [0, 0, 0, 1]. Further, since it may be identified that the patient belongs to the male group, the information on the gender of the patient may be quantified to a vector of [0, 1].

In step S430, the processor 110 may generate the static feature vector which is the multi-hot encoding vector based on the identified group information. For example, the multi-hot encoding vector for male patient A who is 87 years hold and enters the general intensive care unit (MICU) may be forms of [0, 0, 1], [0, 0, 0, 1], and [0, 1] which are vectors expressing the identified group information and a form [0, 0, 1, 0, 0, 0, 1, 0, 1] combined by concatenating three vectors. The multi-hot form vector may be used as the static feature vector in the present disclosure.

In the present disclosure, as a method for driving relationship information between static data groups, an adjacent matrix may be used. The adjacent matrix may be a matrix including information on which relationship vectors have other vectors by computing an inter-vector distance. The adjacent matrix may be pre-generated in a training stage of the artificial neural network, and the adjacent matrix may be generated through the static data and the dynamic data of the training data of the artificial neural network.

The adjacent matrix may compute a dynamic data distribution for a static data group, compute an inter-static data group adjacent degree based on the dynamic data distribution, and may be generated based on comparing the adjacent degree and a predetermined threshold degree. The inter-data group adjacent degree may be measured based on various means including a Wasserstein distance.

For example, it may be assumed that there are three static data groups [A, B, C] for the training data, and there is a distribution [ [symbol] _{A}, [symbol] _{B}, [symbol] _{C}] of the dynamic data for each group. For example, when the training data is data for vital signs of multiple types of patients, the dynamic distribution may be an average value after making standard normal distribution for the vital signs of patients who belong to each group, or a distribution of embedding vectors obtained through the encoder of the machine learning model. The processor 110 may compute a distance between the dynamic data distributions [symbol] _{A} and [symbol] _{B} by using a Wasserstein distance measurement method, and the distance is, that is, defined as a distance between static data groups A and B. The processor 110 may compute a distance between A and C, and a distance between B and C by the same scheme as obtaining the distance between A and B. Thereafter, the processor 110 normalizes the distance between the static data groups to a predetermined criterion, e.g., a value between 0 and 1, and then sets the normalized value as values of elements constituting a matrix to generate an adjacent matrix having a size of 3×3.

As another example, the processor 110 may compute the distances between the respective static data groups, and then convert the respective distances into discrete values through a predetermined threshold, and compute the adjacent matrix by setting the discrete values to the values of the elements constituting the matrix. For example, when the threshold is 0.5, the processor 110 may generate the adjacent matrix by converting each section into the discrete value in such a manner of setting each section to 0 when the distance between the static data groups is 0 or more and less than 0.05, 0.05 when the distance is 0.05 or more and less than 0.1, 0.1 when the distance is 0.1 or more and less than 0.15, 0 when the distance is 0.3 or more and less than 0.5, and 1.0 when the distance is 0.5 or more.

However, in the present disclosure, the method for generating the adjacent matrix is not limited to such an example, and as the method for driving the relationship information between the static data groups, method of other similar purposes other than the adjacent matrix may also be used without a limit.

In FIG. 6, a static data distribution for a plurality of groups is illustrated. In this case, a dark degree of a color represented by a cell of each table may indicate an adjacent degree between two static data groups of a horizontal axis and a vertical axis. The processor 110 may generate the adjacent matrix for the identified group of each static data by using the distribution.

The processor may generate the integrated feature vector based on a multiplication operation result of the static feature vector and the adjacent matrix. Specifically, the processor 110 inputs a vector generated based on the multiplication operation result of the static feature vector and the adjacent matrix into the static data encoder included in the artificial neural network model to generate the integrated feature vector. In the example, with respect to male patient A who is 87 years hold and enters the general intensive care unit (MICU), when the static feature vector is [0, 0, 1, 0, 0, 0, 1, 0, 1], a result of performing a multiplication operation of the static feature vector and the adjacent matrix may be a form of [0.14, 0.85, 0.23, 0.45, 0.55, 0.02, 0.92, 0.09, 0.90]. The processor 110 may determine an output generated by inputting the multiplication operation result into the static data encoder included in the artificial neural network model as the integrated feature vector. The generated integrated feature vector may be a vector to which the relationship information between the static data groups of the input data is reflected.

The processor 110 may generate the final prediction result of the artificial neural network model based on the integrated feature vector and the dynamic feature vector. For example, when the integrated feature vector is [0.24, 0.95, 0.33, 0.55, 0.65, 0.12, 0.02, 0.19, 0.01] and the dynamic feature vector is [0.42, 0.32, 0.56], the processor may generate one vector, i.e., [0.24, 0.95, 0.33, 0.55, 0.65, 0.12, 0.02, 0.19, 0.01, 0.42, 0.32, 0.56] by concatenating the integrated feature vector and the dynamic feature vector. Thereafter, the processor 110 inputs the vector into the classifier included in the artificial neural network to generate an output of the classifier as the final prediction result of the artificial neural network model. However, in the present disclosure the method for generating the final prediction result of the artificial neural network model based on the integrated feature vector and the dynamic feature vector is not limited to the example, and various methods for associating two vectors and another artificial neural network structure of outputting the prediction result based on the feature vector may be used.

In the meantime, according to an embodiment of the present disclosure, a computer readable medium storing a data structure is disclosed.

The data structure may refer to organization, management, and storage of data that enable efficient access and modification of data. The data structure may refer to organization of data for solving a specific problem (for example, data search, data storage, and data modification in the shortest time). The data structure may also be defined with a physical or logical relationship between the data elements designed to support a specific data processing function. A logical relationship between data elements may include a connection relationship between user defined data elements. A physical relationship between data elements may include an actual relationship between the data elements physically stored in a computer readable storage medium (for example, a permanent storage device). In particular, the data structure may include a set of data, a relationship between data, and a function or a command applicable to data. Through the effectively designed data structure, the computing device may perform a calculation while minimally using resources of the computing device. In particular, the computing device may improve efficiency of calculation, reading, insertion, deletion, comparison, exchange, and search through the effectively designed data structure.

The data structure may be divided into a linear data structure and a non-linear data structure according to the form of the data structure. The linear data structure may be the structure in which only one data is connected after one data. The linear data structure may include a list, a stack, a queue, and a deque. The list may mean a series of dataset in which order exists internally. The list may include a linked list. The linked list may have a data structure in which data is connected in a method in which each data has a pointer and is linked in a single line. In the linked list, the pointer may include information about the connection with the next or previous data. The linked list may be expressed as a single linked list, a double linked list, and a circular linked list according to the form. The stack may have a data listing structure with limited access to data. The stack may have a linear data structure that may process (for example, insert or delete) data only at one end of the data structure. The data stored in the stack may have a data structure (Last In First Out, LIFO) in which the later the data enters, the sooner the data comes out. The queue is a data listing structure with limited access to data, and may have a data structure (First In First Out, FIFO) in which the later the data is stored, the later the data comes out, unlike the stack. The deque may have a data structure that may process data at both ends of the data structure.

The non-linear data structure may be the structure in which the plurality of data is connected after one data. The non-linear data structure may include a graph data structure. The graph data structure may be defined with a vertex and an edge, and the edge may include a line connecting two different vertexes. The graph data structure may include a tree data structure. The tree data structure may be the data structure in which a path connecting two different vertexes among the plurality of vertexes included in the tree is one. That is, the tree data structure may be the data structure in which a loop is not formed in the graph data structure.

Throughout the present specification, a calculation model, a nerve network, the network function, and the neural network may be used with the same meaning. Hereinafter, the terms of the calculation model, the nerve network, the network function, and the neural network are unified and described with a neural network. The data structure may include a neural network. Further, the data structure including the neural network may be stored in a computer readable medium. The data structure including the neural network may also include preprocessed data for processing by the neural network, data input to the neural network, a weight of the neural network, a hyper-parameter of the neural network, data obtained from the neural network, an active function associated with each node or layer of the neural network, and a loss function for training of the neural network. The data structure including the neural network may include predetermined configuration elements among the disclosed configurations. That is, the data structure including the neural network may include the entirety or a predetermined combination of pre-processed data for processing by neural network, data input to the neural network, a weight of the neural network, a hyper parameter of the neural network, data obtained from the neural network, an active function associated with each node or layer of the neural network, and a loss function for training the neural network. In addition to the foregoing configurations, the data structure including the neural network may include predetermined other information determining a characteristic of the neural network. Further, the data structure may include all type of data used or generated in a computation process of the neural network, and is not limited to the foregoing matter. The computer readable medium may include a computer readable recording medium and/or a computer readable transmission medium. The neural network may be formed of a set of interconnected calculation units which are generally referred to as “nodes.” The “nodes” may also be called “neurons.” The neural network consists of one or more nodes.

The data structure may include data input to the neural network. The data structure including the data input to the neural network may be stored in the computer readable medium. The data input to the neural network may include training data input in the training process of the neural network and/or input data input to the training completed neural network. The data input to the neural network may include data that has undergone pre-processing and/or data to be pre-processed. The pre-processing may include a data processing process for inputting data to the neural network. Accordingly, the data structure may include data to be pre-processed and data generated by the pre-processing. The foregoing data structure is merely an example, and the present disclosure is not limited thereto.

The data structure may include a weight of the neural network (in the present specification, weights and parameters may be used with the same meaning), Further, the data structure including the weight of the neural network may be stored in the computer readable medium. The neural network may include a plurality of weights. The weight is variable, and in order for the neural network to perform a desired function, the weight may be varied by a user or an algorithm. For example, when one or more input nodes are connected to one output node by links, respectively, the output node may determine a data value output from the output node based on values input to the input nodes connected to the output node and the weight set in the link corresponding to each of the input nodes. The foregoing data structure is merely an example, and the present disclosure is not limited thereto.

For a non-limited example, the weight may include a weight varied in the neural network training process and/or the weight when the training of the neural network is completed. The weight varied in the neural network training process may include a weight at a time at which a training cycle starts and/or a weight varied during a training cycle. The weight when the training of the neural network is completed may include a weight of the neural network completing the training cycle. Accordingly, the data structure including the weight of the neural network may include the data structure including the weight varied in the neural network training process and/or the weight when the training of the neural network is completed. Accordingly, it is assumed that the weight and/or a combination of the respective weights are included in the data structure including the weight of the neural network. The foregoing data structure is merely an example, and the present disclosure is not limited thereto.

The data structure including the weight of the neural network may be stored in the computer readable storage medium (for example, a memory and a hard disk) after undergoing a serialization process. The serialization may be the process of storing the data structure in the same or different computing devices and converting the data structure into a form that may be reconstructed and used later. The computing device may serialize the data structure and transceive the data through a network. The serialized data structure including the weight of the neural network may be reconstructed in the same or different computing devices through deserialization. The data structure including the weight of the neural network is not limited to the serialization. Further, the data structure including the weight of the neural network may include a data structure (for example, in the non-linear data structure, B-Tree, Trie, m-way search tree, AVL tree, and Red-Black Tree) for improving efficiency of the calculation while minimally using the resources of the computing device. The foregoing matter is merely an example, and the present disclosure is not limited thereto.

The data structure may include a hyper-parameter of the neural network. The data structure including the hyper-parameter of the neural network may be stored in the computer readable medium. The hyper-parameter may be a variable varied by a user. The hyper-parameter may include, for example, a learning rate, a cost function, the number of times of repetition of the training cycle, weight initialization (for example, setting of a range of a weight value to be weight-initialized), and the number of hidden units (for example, the number of hidden layers and the number of nodes of the hidden layer). The foregoing data structure is merely an example, and the present disclosure is not limited thereto.

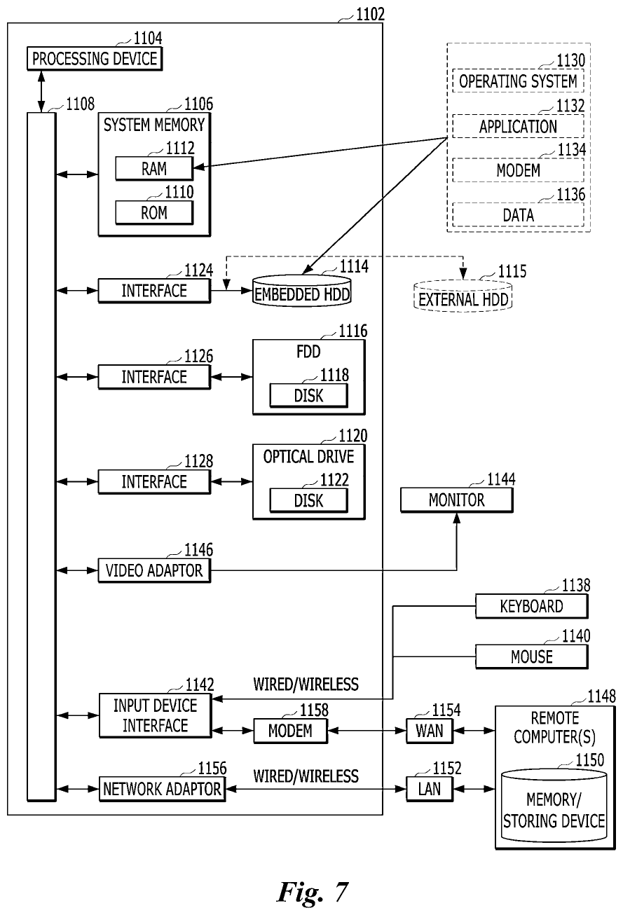

FIG. 7 is a simple and general schematic diagram illustrating an example of a computing environment in which the embodiments of the present disclosure are implementable.

The present disclosure has been described as being generally implementable by the computing device, but those skilled in the art will appreciate well that the present disclosure is combined with computer executable commands and/or other program modules executable in one or more computers and/or be implemented by a combination of hardware and software.

In general, a program module includes a routine, a program, a component, a data structure, and the like performing a specific task or implementing a specific abstract data form. Further, those skilled in the art will well appreciate that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer.

The embodiments of the present disclosure may be carried out in a distribution computing environment, in which certain tasks are performed by remote processing devices connected through a communication network. In the distribution computing environment, a program module may be located in both a local memory storage device and a remote memory storage device.

The computer generally includes various computer readable media. The computer accessible medium may be any type of computer readable medium, and the computer readable medium includes volatile and non-volatile media, transitory and non-transitory media, and portable and non-portable media. As a non-limited example, the computer readable medium may include a computer readable storage medium and a computer readable transport medium. The computer readable storage medium includes volatile and non-volatile media, transitory and non-transitory media, and portable and non-portable media constructed by a predetermined method or technology, which stores information, such as a computer readable command, a data structure, a program module, or other data. The computer readable storage medium includes a RAM, a Read Only Memory (ROM), an Electrically Erasable and Programmable ROM (EEPROM), a flash memory, or other memory technologies, a Compact Disc (CD)-ROM, a Digital Video Disk (DVD), or other optical disk storage devices, a magnetic cassette, a magnetic tape, a magnetic disk storage device, or other magnetic storage device, or other predetermined media, which are accessible by a computer and are used for storing desired information, but is not limited thereto.

The computer readable transport medium generally implements a computer readable command, a data structure, a program module, or other data in a modulated data signal, such as a carrier wave or other transport mechanisms, and includes all of the information transport media. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, Radio Frequency (RF), infrared rays, and other wireless media. A combination of the predetermined media among the foregoing media is also included in a range of the computer readable transport medium.

An illustrative environment 1100 including a computer 1102 and implementing several aspects of the present disclosure is illustrated, and the computer 1102 includes a processing device 1104, a system memory 1106, and a system bus 1108. The system bus 1108 connects system components including the system memory 1106 (not limited) to the processing device 1104. The processing device 1104 may be a predetermined processor among various commonly used processors. A dual processor and other multi-processor architectures may also be used as the processing device 1104.

The system bus 1108 may be a predetermined one among several types of bus structure, which may be additionally connectable to a local bus using a predetermined one among a memory bus, a peripheral device bus, and various common bus architectures. The system memory 1106 includes a ROM 1110, and a RAM 1112. A basic input/output system (BIOS) is stored in a non-volatile memory 1110, such as a ROM, an EPROM, and an EEPROM, and the BIOS includes a basic routing helping a transport of information among the constituent elements within the computer 1102 at a time, such as starting. The RAM 1112 may also include a high-rate RAM, such as a static RAM, for caching data.

The computer 1102 also includes an embedded hard disk drive (HDD) 1114 (for example, enhanced integrated drive electronics (EIDE) and serial advanced technology attachment (SATA))—the embedded HDD 1114 being configured for exterior mounted usage within a proper chassis (not illustrated)—a magnetic floppy disk drive (FDD) 1116 (for example, which is for reading data from a portable diskette 1118 or recording data in the portable diskette 1118), and an optical disk drive 1120 (for example, which is for reading a CD-ROM disk 1122, or reading data from other high-capacity optical media, such as a DVD, or recording data in the high-capacity optical media). A hard disk drive 1114, a magnetic disk drive 1116, and an optical disk drive 1120 may be connected to a system bus 1108 by a hard disk drive interface 1124, a magnetic disk drive interface 1126, and an optical drive interface 1128, respectively. An interface 1124 for implementing an outer mounted drive includes, for example, at least one of or both a universal serial bus (USB) and the Institute of Electrical and Electronics Engineers (IEEE) 1394 interface technology.

The drives and the computer readable media associated with the drives provide non-volatile storage of data, data structures, computer executable commands, and the like. In the case of the computer 1102, the drive and the medium correspond to the storage of random data in an appropriate digital form. In the description of the computer readable media, the HDD, the portable magnetic disk, and the portable optical media, such as a CD, or a DVD, are mentioned, but those skilled in the art will well appreciate that other types of computer readable media, such as a zip drive, a magnetic cassette, a flash memory card, and a cartridge, may also be used in the illustrative operation environment, and the predetermined medium may include computer executable commands for performing the methods of the present disclosure.

A plurality of program modules including an operation system 1130, one or more application programs 1132, other program modules 1134, and program data 1136 may be stored in the drive and the RAM 1112. An entirety or a part of the operation system, the application, the module, and/or data may also be cached in the RAM 1112. It will be well appreciated that the present disclosure may be implemented by several commercially usable operation systems or a combination of operation systems.

A user may input a command and information to the computer 1102 through one or more wired/wireless input devices, for example, a keyboard 1138 and a pointing device, such as a mouse 1140. Other input devices (not illustrated) may be a microphone, an IR remote controller, a joystick, a game pad, a stylus pen, a touch screen, and the like. The foregoing and other input devices are frequently connected to the processing device 1104 through an input device interface 1142 connected to the system bus 1108, but may be connected by other interfaces, such as a parallel port, an IEEE 1394 serial port, a game port, a USB port, an IR interface, and other interfaces.

A monitor 1144 or other types of display devices are also connected to the system bus 1108 through an interface, such as a video adaptor 1146. In addition to the monitor 1144, the computer generally includes other peripheral output devices (not illustrated), such as a speaker and a printer.

The computer 1102 may be operated in a networked environment by using a logical connection to one or more remote computers, such as remote computer(s) 1148, through wired and/or wireless communication. The remote computer(s) 1148 may be a work station, a computing device computer, a router, a personal computer, a portable computer, a microprocessor-based entertainment device, a peer device, and other general network nodes, and generally includes some or an entirety of the constituent elements described for the computer 1102, but only a memory storage device 1150 is illustrated for simplicity. The illustrated logical connection includes a wired/wireless connection to a local area network (LAN) 1152 and/or a larger network, for example, a wide area network (WAN) 1154. The LAN and WAN networking environments are general in an office and a company, and make an enterprise-wide computer network, such as an Intranet, easy, and all of the LAN and WAN networking environments may be connected to a worldwide computer network, for example, the Internet.

When the computer 1102 is used in the LAN networking environment, the computer 1102 is connected to the local network 1152 through a wired and/or wireless communication network interface or an adaptor 1156. The adaptor 1156 may make wired or wireless communication to the LAN 1152 easy, and the LAN 1152 also includes a wireless access point installed therein for the communication with the wireless adaptor 1156. When the computer 1102 is used in the WAN networking environment, the computer 1102 may include a modem 1158, is connected to a communication computing device on a WAN 1154, or includes other means setting communication through the WAN 1154 via the Internet. The modem 1158, which may be an embedded or outer-mounted and wired or wireless device, is connected to the system bus 1108 through a serial port interface 1142. In the networked environment, the program modules described for the computer 1102 or some of the program modules may be stored in a remote memory/storage device 1150. The illustrated network connection is illustrative, and those skilled in the art will appreciate well that other means setting a communication link between the computers may be used.

The computer 1102 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes a wireless fidelity (Wi-Fi) and Bluetooth wireless technology at least. Accordingly, the communication may have a pre-defined structure, such as a network in the related art, or may be simply ad hoc communication between at least two devices.

The Wi-Fi enables a connection to the Internet and the like even without a wire. The Wi-Fi is a wireless technology, such as a cellular phone, which enables the device, for example, the computer, to transmit and receive data indoors and outdoors, that is, in any place within a communication range of a base station. A Wi-Fi network uses a wireless technology, which is called IEEE 802.11 (a, b, g, etc.) for providing a safe, reliable, and high-rate wireless connection. The Wi-Fi may be used for connecting the computer to the computer, the Internet, and the wired network (IEEE 802.3 or Ethernet is used). The Wi-Fi network may be operated at, for example, a data rate of 11 Mbps (802.11a) or 54 Mbps (802.11b) in an unauthorized 2.4 and 5 GHz wireless band, or may be operated in a product including both bands (dual bands).

Those skilled in the art may appreciate that information and signals may be expressed by using predetermined various different technologies and techniques. For example, data, indications, commands, information, signals, bits, symbols, and chips referable in the foregoing description may be expressed with voltages, currents, electromagnetic waves, magnetic fields or particles, optical fields or particles, or a predetermined combination thereof.

Those skilled in the art will appreciate that the various illustrative logical blocks, modules, processors, means, circuits, and algorithm operations described in relationship to the embodiments disclosed herein may be implemented by electronic hardware (for convenience, called “software” herein), various forms of program or design code, or a combination thereof. In order to clearly describe compatibility of the hardware and the software, various illustrative components, blocks, modules, circuits, and operations are generally illustrated above in relation to the functions of the hardware and the software. Whether the function is implemented as hardware or software depends on design limits given to a specific application or an entire system. Those skilled in the art may perform the function described by various schemes for each specific application, but it shall not be construed that the determinations of the performance depart from the scope of the present disclosure.

Various embodiments presented herein may be implemented by a method, a device, or a manufactured article using a standard programming and/or engineering technology. A term “manufactured article” includes a computer program, a carrier, or a medium accessible from a predetermined computer-readable storage device. For example, the computer-readable storage medium includes a magnetic storage device (for example, a hard disk, a floppy disk, and a magnetic strip), an optical disk (for example, a CD and a DVD), a smart card, and a flash memory device (for example, an EEPROM, a card, a stick, and a key drive), but is not limited thereto. Further, various storage media presented herein include one or more devices and/or other machine-readable media for storing information.

It shall be understood that a specific order or a hierarchical structure of the operations included in the presented processes is an example of illustrative accesses. It shall be understood that a specific order or a hierarchical structure of the operations included in the processes may be rearranged within the scope of the present disclosure based on design priorities. The accompanying method claims provide various operations of elements in a sample order, but it does not mean that the claims are limited to the presented specific order or hierarchical structure.

The description of the presented embodiments is provided so as for those skilled in the art to use or carry out the present disclosure. Various modifications of the embodiments may be apparent to those skilled in the art, and general principles defined herein may be applied to other embodiments without departing from the scope of the present disclosure. Accordingly, the present disclosure is not limited to the embodiments suggested herein, and shall be interpreted within the broadest meaning range consistent to the principles and new characteristics presented herein.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method performed by a computing device to generate a prediction result by using static data and dynamic data, the method comprising:
generating an integrated feature vector from static data and dynamic data of input data by using an artificial neural network model;
generating a dynamic feature vector from the dynamic data of the input data by using the artificial neural network model; and
generating a final prediction result of the artificial neural network model based on the integrated feature vector and the dynamic feature vector.

2. The method of claim 1, wherein the dynamic data includes time-series biometric data, and
wherein the static data includes information related to a patient other than the time-series biometric data.

3. The method of claim 1, wherein the generating of the integrated feature vector includes:
identifying a group to which the static data of the input data belongs;
generating a static feature vector from the static data of the input data;
obtaining an inter-group adjacent matrix based on dynamic data information corresponding to an identified static data group; and
generating the integrated feature vector based on a computation of the static feature vector and the adjacent matrix.

4. The method of claim 3, wherein the identifying of the group to which the static data of the input data belongs includes:
categorizing information included in the static data; and
identifying one or more groups to which the static data belongs based on the categorized information.

5. The method of claim 3, wherein the generating of the static feature vector from the static data of the input data includes:
generating a multi-hot encoding vector based on identified group information.

6. The method of claim 3, wherein the adjacent matrix is generated based on:
computing a dynamic data distribution for dynamic data corresponding to the identified static data group;
computing a distance between static data groups based on the dynamic data distribution; and
comparing the distance between the static data groups and a predetermined threshold distance.

7. The method of claim 1, wherein the generating of the dynamic feature vector from the dynamic data of the input data by using the artificial neural network model includes:
preprocessing the dynamic data of the input data; and
generating a dynamic feature vector based on the preprocessed dynamic data.

8. A computer program stored in a non-transitory computer-readable storage medium, wherein the computer program causes at least one processor to perform operations of generating a prediction result by using static data and dynamic data, and the operations include:
an operation of generating an integrated feature vector from static data and dynamic data of input data by using an artificial neural network model;
an operation of generating a dynamic feature vector from the dynamic data of the input data by using the artificial neural network model; and
an operation of generating a final prediction result of the artificial neural network model based on the integrated feature vector and the dynamic feature vector.

9. A computing device comprising:
at least one processor; and
a memory coupled to the at least one processor,
wherein the at least one processor is configured to:
generate an integrated feature vector from static data and dynamic data of input data by using an artificial neural network model;
generate a dynamic feature vector from the dynamic data of the input data by using the artificial neural network model; and
generate a final prediction result of the artificial neural network model based on the integrated feature vector and the dynamic feature vector.
